(19) United States
(12) Patent Application Publication
(10) Pub. No.: US 2023/0292806 A1
(43) Pub. Date: Sep. 21, 2023

(54) COMPOSITIONS AND METHODS OF PRODUCTION THEREOF

(71) Applicant: Optibiotix Limited, York (GB)

(72) Inventors: Stephen Patrick O'Hara, York (GB); Sofia Kolida, York (GB)

(21) Appl. No.: 18/041,485

(22) PCT Filed: Aug. 13, 2021
(86) PCT No.: PCT/GB2021/052114

§ 371 Date: Feb. 13, 2023

(30) Foreign Application Priority Data
Aug. 14, 2020 (GB) 2012765.0

(51) Int. Cl. A23L 33/21; A23L 33/125; A23L 33/16; A61K 33/24; A61K 31/702

(52) U.S. Cl. CPC A23L 33/21; A23L 33/125; A23L 33/16; A61K 33/24; A61K 31/702

(57) ABSTRACT

The present invention relates to a composition comprising prebiotic, chromium and soluble fibre components, wherein the composition is formed by the agglomeration of the components into one or more granules. The invention also relates to methods of producing the one or more granules.

### TECHNICAL FIELD OF THE INVENTION

The invention relates to compositions for weight management that support health & wellbeing by promoting the diversity of the gut microbiome and methods of production thereof.

### BACKGROUND TO THE INVENTION

Overweight and obese are conditions defined as abnormal or excessive fat accumulation that may impair health. It results from imbalances in the body's regulation of energy intake, expenditure and storage.

Obesity is one of the greatest public health challenges of the 21st century. It is a complex condition, one with serious social and psychological dimensions, that affects virtually all age and socioeconomic groups in both developed and developing countries. The health consequences of obesity range from increased risk of premature death to serious chronic conditions that reduce the overall quality of life.

Soluble dietary fibres and prebiotics have attracted interest as candidate compounds for the control of obesity and associated metabolic disorders. In animal studies, prebiotics have been shown to regulate the intake of food, prevent weight gain, beneficially alter lipid metabolism and reduce obesity-related inflammation. So far, most studies have focused on simply supplementing the diet with inulin and fructooligosaccharides (FOS).

Prebiotics are dietary ingredients which can increase the diversity of the gut microbiome. They can selectively enhance beneficial indigenous gut microbiota, such as lactobacilli or bifidobacteria, and are finding much increased application into the food sector. Prebiotics are non digestible food ingredients that are selectively metabolised by colonic bacteria which contribute to improved health. As such, their use can promote beneficial changes within the indigenous gut microbial milieu and they can therefore help survivability of probiotics.

Although obesity is caused by an excess caloric intake which is not matched by an increase in energy expenditure, differences in gut microbiota composition and activities between individuals may also be an important contributing factor affecting energy homeostasis. This would imply that the gut health and the gut microbiota of obese individuals would be more efficient in salvaging and/or storing energy from a specific diet compared to the microbiota of lean individuals.

Some dietary fibres can form viscous gels on exposure to an aqueous environment and their gelling properties may account for weight loss promoting effects by delaying gastric emptying, slowing bowel transit time and blunting post-prandial surges in insulin and glucose.

WO2015/067936 discloses compositions for weight management comprising: a) a microbiome modifying component; b) a satiety modifying component; and c) a metabolic modifying component. More specifically, the document exemplifies a composition comprising a prebiotic, a soluble fibre (glucomannan) and chromium. The composition is currently marketed under the Slimbiome®, Optibiome®, and WellBiome® brands by Optibiotix Limited, UK and have proven to be highly successful commercial products with a number of applications, with inclusion in meal replacement shakes, snack bars and cereals and protein bar products. However, the formulation of the composition has posed a number of challenges in terms of product uniformity, solubility, dispersion, storage and ease of downstream processing. These can impact on the taste, texture, shelf life and quality control of finished products.

It is therefore an object of the present invention to provide a formulation of a prebiotic, a soluble fibre and chromium (and associated methods of production) which can address one or more of the above identified problems, providing more consistent product presentation, improved flavour and mouthfeel. It would be preferred if the resultant formulation has high uniformity and dispersion of the components when consumed. It would also be desirable if the formulation was stable and had a long shelf life. Furthermore, it would be beneficial if the formulation were easy to transport and easy to formulate into a range of diverse products.

### SUMMARY OF THE INVENTION

In accordance with a first aspect of the present invention, there is provided a composition comprising prebiotic, chromium and soluble fibre components, wherein the composition is formed by the agglomeration of the components into one or more granules.

The term “prebiotic” is intended to mean a selectively fermented ingredient that allows specific changes, both in the composition and/or activity in the gastrointestinal microflora flora that confers benefits upon host wellbeing and health. Prebiotics act in the colon and produce changes in the microbial flora which affect energy metabolism and gut peptides involved in satiation (GLP1, GLP2) and can have a long acting affect.

Chromium acts systemically potentiating insulin action, increasing metabolic rate, influencing carbohydrate, lipid and protein metabolism and maintaining glucose levels.

The soluble fibre may be selected from a number of soluble fibres which act primarily in the stomach by suppressing appetite by gel formation in the stomach, causing distension and activating mechanoreceptors that signal increased satiety and fullness. The presence of a gel may also prolong the release of stomach contents into the small intestine reducing the rate at which carbohydrate is absorbed by the small intestine moderating blood glucose levels.

The soluble fibre will preferably be selected from one or more of the following: Beta glucans, pectin, gums, psyllium and algin. Most preferably, the soluble fibre will comprise glucomannan.

The composition will preferably be formed by the wet agglomeration of the components. The agglomeration of the components may be batch or continuous by means of a fluid bed agglomerator. The fluid bed agglomerator may utilise a number of different materials as binding agents including various solutions and/or simply water. It is preferred that the binding agent is water which is atomised within the fluid bed agglomerator.

The granule may have a diameter of about 2 mm or less. However, the granule may have a diameter of about 1.9 mm or less, about 1.8 mm or less, about 1.7 mm or less, about 1.6 mm or less, about 1.5 mm or less, about 1.4 mm or less, about 1.3 mm or less, about 1.2 mm or less, about 1.1 mm or less and most preferably about 1 mm or less.

Alternatively, the granule may have a diameter in the range of about 50 μm to about 2 mm. However, the granule may have a diameter of about 60 μm to about 1.75 mm, about 70 μm to about 1.5 mm, about 80 μm to about 1.25 mm and most preferably about 90 μm to about 1 mm.

The granule may have a moisture content of about 10% or less. However, the granule may have a moisture content of about 9.5% or less, about 8% or less, about 7.5% or less, about 7% or less, about 6.5% or less, about 6% or less, about 5.5% and most preferably about 5% or less.

The prebiotic component may comprise one or more selected from: inulin, fructooligosaccharides (FOS) (including but not limited to FOS produced sucrose using fructosyl transferases), galactooligosaccharides (GOS), α-galactooligosaccharides (α-GOS), α-gluco-oligosaccharides, xylooligosaccharides (XOS), gentiooligosaccharides and combinations thereof. Preferably, the prebiotic component comprises fructooligosaccharides (FOS) and in particular fructooligosaccharides (FOS) derived from inulin by partial enzymatic hydrolysis. Preferably, the fructooligosaccharides (FOS) consists of oligosaccharides which are based on fructose units linked together by μ(2-1) linkages. Part of the molecules may be terminated by a glucose unit. The fructooligosaccharides (FOS) has a total number of fructose or glucose units in the range of about 2 and about 8. Preferably, the fructooligosaccharides (FOS) has a purity of about 95% or more.

As chromium (III) as chromium chloride or chromium nicotinate are poorly absorbed, it is preferred that the chromium component comprises chromium picolinate. It is also preferable, that the chromium picolinate comprises about 12% or more pure chromium.

The chromium component may have a particle size of about 250 μm or less, about 225 μm or less, about 200 μm or less, about 190 μm or less, but most preferably about 180 μm or less or about 177 μm or less.

The soluble fibre component may have a particles size of about 175 μm or less, about 150 μm or less, about 140 μm or less, about 150 μm or less, about 140 μm or less, about 135 μm or less, about 130 μm or less and most preferably 125 μm or less.

The composition may have a moisture content of about 10% or less, about 9.5% or less, about 9% or less, about 8.5% or less, about 8% or less, about 7.5% or less, about 7% or less, about 7% or less, about 6.5% or less, about 6.0% or less, about 5.5% or less, but most preferably about 5% or less.

The composition preferably comprises a plurality of granules. Advantageously, the plurality of granules have been found to form highly stable and easily dispersible powder having excellent flow properties, enabling easy transportation and onward blending with other ingredients.

The plurality of granules may comprise a bulk density in the range of about 0.4 g/cc to about 0.8 g/cc, about 0.45 g/cc to about 0.75 g/cc, about 0.5 g/cc to about 0.7 g/cc, but most preferably about 0.55 g/cc to about 0.65 g/cc.

It will be apparent to the skilled addressee that the granules may be formed having a number of preferred diameters.

In certain embodiments, the granules have a diameter of less than about 2 mm, less than about 1.95 mm, less than about 1.90 mm, less than about 1.85 mm, less than about 1.80 mm, less than about 1.75 mm, less than about 1.70 mm, less than about 1.65 mm, less than about 1.60 mm, less than about 1.55 mm, but most preferably less than about 1.5 mm.

In other embodiments, it is preferred that less than about 20% of the granules may be about 1 mm or greater in diameter and/or less than about 20% of the granules may be about 90 μm or less in diameter. Less than about 15% of the granules may be about 1 mm or greater in diameter and/or less than about 15% of the granules may be about 90 μm or less in diameter. Most preferably, less than about 10% of the granules may be about 1 mm or greater in diameter and/or less than about 10% of the granules may be about 90 μm or less in diameter.

The composition may be for use in in a number of different applications—such as for incorporation into a food-stuff or as a food additive or as a food supplement, or medical device. The composition may be for use as a dietary and/or weight loss supplement and to improve gut microbiome composition and activity. The composition may be for use in the weight management (including reducing overall mass) in an individual. Alternatively, the composition may be for use as a medicament. As a medicament, the composition may be for use in the prevention, management and/or treatment of obesity and/or related health conditions.

Depending on the application of the composition, it may be blended or mixed with other ingredients such as protein of fruit, fruit purees, or concentrates so as to form a foodstuff or other consumable or therapeutic product.

The composition may further comprise or be blended or mixed with an excipient, carrier or gelling compound to modify the release profile of one or more of the components through the intestinal environment. Alternatively, the composition may be blended or mixed with a food stuff, beverage or food additive. The composition could be used directly in granular form as a dietary supplement—for example to be blended by the consumer with foods/drinks or consumed alongside or within foods/drinks.

The composition may be blended or mixed with one or more active ingredients selected from but not limited to: proteins, prebiotics, probiotics, vitamins, minerals, dietary fibres (beta-glucans, gums, psyllium, algin, resistant starch, arabinoxylans, xylans, pectin, polydextrose, chitosans and derivatives thereof), phytochemicals, antioxidants, and combinations thereof.

Vitamins may include fat soluble vitamins such as vitamin A, vitamin D, vitamin E, and vitamin and combinations thereof. In some embodiments, vitamins can include water soluble vitamins such as vitamin C (ascorbic acid), the B vitamins (thiamine or B 1, riboflavoin or B25 niacin or B3, pyridoxine or B6, folic acid or B9, cyanocobalimin or B12, pantothenic acid, biotin), and combinations thereof.

Minerals may include, but are not limited to, sodium, magnesium, chromium, iodine, iron, manganese, calcium, copper, fluoride, potassium, phosphorous, molybdenum, selenium, zinc, and combinations thereof.

Antioxidants may include but are not limited to ascorbic acid, citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, fiavonoids, and combinations thereof.

Phytochemicals may include but are not limited to cartotenoids, chlorophyll, chlorophyllin, fiber, flavanoids, anthocyamns, cyaniding, delphinidin, malvidin, pelargonidin, peonidin, petunidin, flavanols, catechin, epicatechin, epigallocatechin, epigailocatechingallate, theaflavins, thearubigins, proanthocyanins, flavonols, quercetin, kaempferol, myricetin, isorhamnetin, flavononeshesperetin, naringenin, eriodictyol, tangeretin, flavones, apigenin, luteolin, lignans, phytoestrogens, resveratrol, isoflavones, daidzein, genistein, glycitein, soy isoflavones, and combinations thereof.

In accordance with a further aspect of the present invention, there is provided a method of producing a composition comprising prebiotic, chromium and soluble fibre components, wherein the composition is produced by mixing together prebiotic, chromium and soluble fibre components under conditions effective to enable agglomeration of the components into one or more granules.

It is preferred that the prebiotic, chromium and soluble fibre components are mixed under conditions so as to enable wet agglomeration of the components. Such mixing will preferably be conducted in a fluid bed agglomerator. In order to effect agglomeration, the prebiotic, chromium and soluble fibre components may be wetted prior to, during or after mixing. A number of wetting agents can be used from specialised ingestible wetting solutions or simply water. It is preferred that water is employed as the binding agent. The wetting agent is preferably atomised using an atomiser. The method preferably employs multiple fluidisation and drying cycles so as to build up the granules in a consistent manner. This helps to produce a consistently sized granules and also increases granule strength which assists with storage, transportation and subsequent processing into a final product.

The resultant one or more granules will preferably comprise a substantially homogenous mixture of the prebiotic, chromium and soluble fibre components.

The prebiotic component may be provided in a powdered form.

The chromium component may be provided in a powdered form with a particle size of about 250 μm or less, about 225 μm or less, about 200 μm or less, about 190 μm or less, but most preferably about 180 μm or less or about 177 μm or less.

The soluble fibre component may be provided in a powdered form with a particles size of about 175 μm or less, about 150 μm or less, about 140 μm or less, about 150 μm or less, about 140 μm or less, about 135 μm or less, about 130 μm or less and most preferably 125 μm or less.

The method can be employed to produce a composition as herein above described. The method may be employed to produce a plurality of granules. The plurality of granules formed may have a bulk density in the range of about 0.40 g/cc to about 0.8 g/cc, in the range of about 0.45 g/cc to about 0.75 g/cc, in the range of about 0.50 g/cc to about 0.70 g/cc, but preferably in the range of about 0.55 g/cc to about 0.65 g/cc.

The method may further comprise the steps of reducing the moisture content of the granules to about 10% or less, about 9.5% or less, about 9% or less, about 8.5% or less, about 8% or less, about 7.5% or less, about 7% or less, about 7% or less, about 6.5% or less, about 6.0% or less, about 5.5% or less, but most preferably about 5% or less. Reducing the moisture content of the granules may be effected a number of ways, such as drying using headed air.

The method will preferably be run on a batch basis so as to granules having a certain diameter. If required, the granules may be sieved or filtered so that the resultant powder only contains granules having a diameter of less than about 2 mm, less than about 1.95 mm, less than about 1.90 mm, less than about 1.85 mm, less than about 1.80 mm, less than about 1.75 mm, less than about 1.70 mm, less than about 1.65 mm, less than about 1.60 mm, less than about 1.55 mm, but most preferably less than about 1.5 mm.

The method has been shown to advantageously provide for the components to be consistently homogenously dispersed throughout each granule. The method also increases the surface area of each granule which therefore provides for a powder for having improved dispersion characteristics of the components, which is particularly important for soluble fibres such as glucomannan.

### DETAILED DESCRIPTION OF THE INVENTION

Embodiments of the present invention will now be described, by way of example and with reference to the following figures which show:

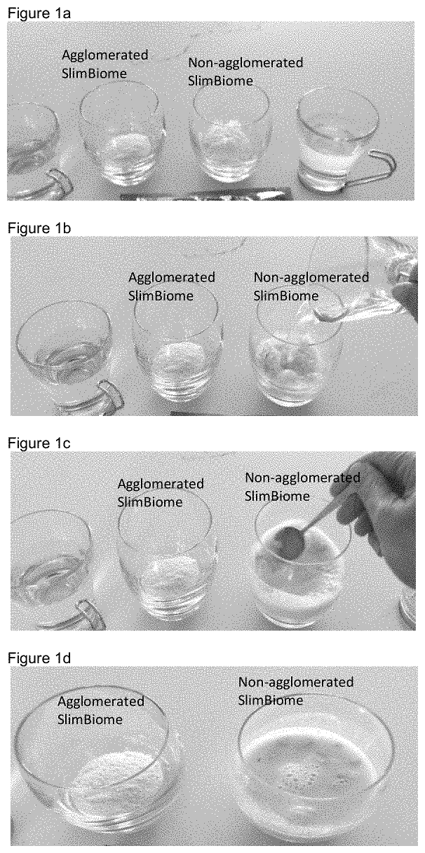

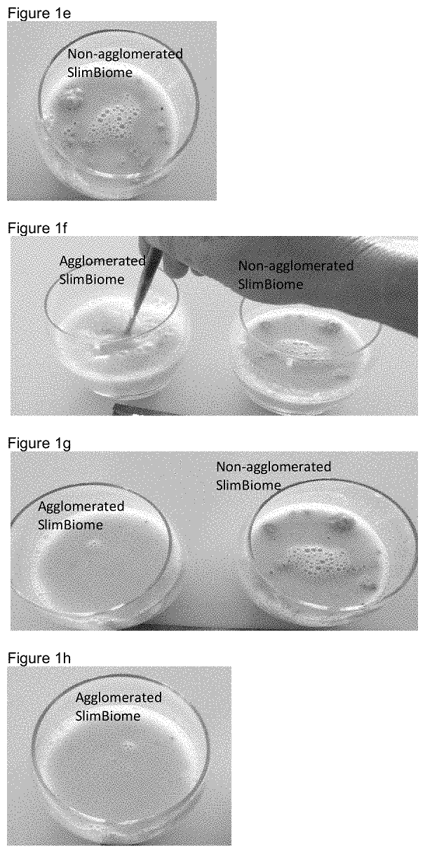

FIG. 1a: Dry samples of agglomerated and non-agglomerated Slimbiome®

FIG. 1b: Water addition to a non-agglomerated Slimbiome® sample.

FIG. 1c: Stirring of the non-agglomerated Slimbiome® sample in water.

FIG. 1d: Non-agglomerated Slimbiome® sample dispersed in water.

FIG. 1e: Non-agglomerated Slimbiome® sample dispersed in water.

FIG. 1f: Stirring of agglomerated Slimbiome® following water addition.

FIG. 1g: Comparison of agglomerated and non-agglomerated Slimbiome® samples dispersed in water.

FIG. 1h: Agglomerated Slimbiome® sample dispersed in water,

### Example 1—Production of Granular Composition Comprising FOS, Chromium and Glucomannan

A granular composition was produced using the technique of fluid bed batch agglomeration to produce granules comprising FOS, chromium and glucomannan.

Prior to mixing, the FOS, chromium and glucomannan were all provided in powder form.

The FOS was in the form of a powder containing mainly oligofructose produced by partial enzymatic hydrolysis of chicory inulin and consisted of oligosaccharides which were based on fructose units linked together by β(2-1) linkages. Part of the molecules were terminated by a glucose unit. The total number of fructose or glucose units (=Degree of Polymerisation or DP) ranged mainly between 2 and 8.

The chromium was chromium picolinate in the form of a powder with a standardised content of 12% pure chromium. The chromium picolinate powder had a moisture content of less than 5% and a particle size of 99-100% through 80 mesh (177 μm).

The glucomannan was in a powdered form and was obtained from the konjac flour of various species of Amorphophallus by aqueous extraction. The glucomannan had a particle size of at least 90% being able to pass through 120-200 mesh (125 μm-74 μm).

Initially 0.04 kg of chromium picolinate powder is pre-blended with 1 kg of FOS before being loaded on to the fluid bed of the fluid bed batch agglomeration apparatus along with the glucomannan and the powders mixed in a fluidisation stage, where heated air is blown from underneath the fine mesh bed gauze so as to lift, mix and move the powder so that it could come into contact with atomised water During the spraying stage, the mixed powder is wetted via air atomised cold water (or solution) so as to start the agglomeration of the particles of the powders into small granules as the particles subsequently sticks together. The fluidisation cycles the powders so that they are wetting and drying and gradually building up strength into a granule as it increases with size. During the drying stage, the heated air is used to dry off the water which adds to granule strength. The fluid bed batch agglomeration is run until the granules have reached the desired diameter of about 1.5 mm and if required the granules are sieved through a 1.5 mm screen so as remove larger granules.

Different agglomeration durations were assessed. The length of the process is typically dictated by the amount of water sprayed on to the powder and thus the amount of moisture that needs to be dried off. Typically, the longer the process the better the agglomeration and the more consistent particle size distribution achieved. However, a longer agglomeration process is more expensive. A short, long and mid duration process all showed excellent dispersion characteristics compared to a control. The process duration selected was based on a cost benefit analysis. The duration selected achieved a product with good solubility and dispersion characteristics balanced by cost considerations. The granules form a beige coloured free flowing powder having a desirable bulk density of between 0.55 to 0.65 g/cc with a moisture content of 5% or less. The granules enable a homogenous granule of the components which greatly assist in the delivery of the components when formulated with foodstuffs and/or ingested.

When stored in a cool, dry, odour free environment, and not in contact with the floor and in ambient storage conditions, the product advantageously has a shelf life of at least 24 months.

Furthermore, the agglomeration increases the powder's surface area thus improving its dispersion characteristics, which is particularly relevant for glucomannan.

### Example 2—Analysis of Different Slimbiome® Batches

Tables 1 and 2 below show the chromium and soluble fibre contents of agglomerated Slimbiome® samples at two different mesh sizes.

TABLE 1
Slimbiome ® 160 mesh, uncooled sample,
chromium and dietary fibre content

| Lab | TC | Analysis | Unit | Result |
|---|---|---|---|---|
| Q EUNLHE | HEM61 | Chromium | | |
| | | (ICP-MS) | | |
| | | Internal Method | | |
| | | Chromium (Cr) | mg/kg | 8.1 |
| EUNLHE | HEC1A | Dietary fibre | | |
| | | HMWDF & | | |
| | | LMWDF | | |
| | | Internal Method | | |
| | | HMWDF + RS | % (w/w) | 11.3 |
| | | LMWDF | % (w/w) | 73.3 |
| | | Total dietary | % (w/w) | 84.6 |
| | | fibre | | |

TABLE 2
Slimbiome ® 250 mesh, uncooled sample,
chromium and dietary fibre content

| Lab | TC | Analysis | Unit | Result |
|---|---|---|---|---|
| Q EUNLHE | HEM61 | Chromium (ICP- | | |
| | | MS) Internal | | |
| | | Method | | |
| | | Chromium (Cr) | mg/kg | 9.7 |
| EUNLHE | HEC1A | Dietary fibre | | |
| | | HMWDF & | | |
| | | LMWDF Internal | | |
| | | Method | | |
| | | HMWDF + RS | % (w/w) | 12.7 |
| | | LMWDF | % (w/w) | 87.2 |
| | | Total dietary fibre | % (w/w) | 99.9 |

Advantageously agglomeration aids the mixing process, especially for small volume additions, as in the case of chromium. Adding mg quantities to kg quantities can result in inadequate mixing and variation in the concentration of constituents throughout the mix. This raises potential issues with product performance. The results in Tables 1 and 2 for the two Slimbiome® products demonstrate consistency in the concentration of their constituent parts. Without agglomeration existing industry processes would be unable to consistently provide adequate mixing leading to costly quality control (QC) failures and possible poor product performance. At worse, if dispersal was uneven and high levels of chromium were consumed it could lead to unwanted side effects.

### Example 3—Solubility Testing of Non-Agglomerated and Agglomerated Slimbiome®

The solubility of identical quantities of non-agglomerated and agglomerated Slimbiome® were mixed in identical quantities of water as shown in FIGS. 1a-1h.

FIGS. 1d-1h demonstrate the increased water solubility of an agglomerated Slimbiome® formulation. Typically, the raw materials of Slimbiome® are insoluble in cold water. Glucomannan presents difficulties as it swells, gels and thickens upon hydration and, importantly, it requires constant and vigorous agitation for it to dissolve. Advantageously agglomeration provides the ideal process to improve Slimbiome®'s functionality as it increases surface area and produces a consistent bulk density and particle size.

FIG. 1e shows visible lumps of non-agglomerated Slimbiome® remaining upon dispersion in water. In contrast FIG. 1h shows no visible lumps remaining after dispersion of agglomerated Slimbiome® in water. The figures demonstrate the superior water solubility of agglomerated Slimbiome® compared to non-agglomerated Slimbiome®.

The agglomerated Slimbiome® product can be formulated in a number of formats, such as a pharmaceutical powder for inclusion in a tablet or as a soluble powder which is hydrated prior to admission. The Slimbiome® product can also be formulated as a dietary supplement or meal replacement, such as a milkshake or bar or into porridge, muesli, or healthy snacks like fruit gummies.

The forgoing embodiments are not intended to limit the scope of the protection afforded by the claims, but rather to describe examples of how the invention may be put into practice.

1. A composition comprising two or more prebiotics, chromium and soluble fibre components, wherein the composition is formed by the physical binding together of the components into a powder with an average particle size of less than or equal to about 2 mm.

2. The composition as claimed in claim 1, wherein the composition is formed by the wet agglomeration of the components.

3. The composition as claimed in either claim 1 or 2, wherein the agglomeration of the components is by batch or continuous by means of a fluid bed agglomerator.

4. The composition as claimed in claim 3, wherein the fluid bed agglomerator utilises water as a binding agent.

5. The composition as claimed in any preceding claim, wherein the average particle size is of about 1.5 mm or less.

6. The composition as claimed in claim 5, wherein the average particle size is of about 1 mm or less.

7. The composition as claimed in any one of claims 1 to 4, wherein the average particle size is in the range of about 50 μm to about 2 mm.

8. The composition as claimed in claim 7, wherein the average particle size is in the range of about 90 μm to about 1 mm.

9. The composition as claimed in any preceding claim, wherein the prebiotic components comprises two or more selected from: inulin, fructooligosaccharides (FOS), galactooligosaccharides (GOS), α-galactooligosaccharides (α-GOS), α-gluco-oligosaccharides, xylooligosaccharides (XOS), gentiooligosaccharides and combinations thereof.

10. The composition as claimed in claim 9, wherein the prebiotic component comprises fructooligosaccharides (FOS).

11. The composition as claimed in claim 10, wherein the fructooligosaccharides (FOS) is derived from chicory.

12. The composition as claimed in claim 10 or 11, wherein the fructooligosaccharides (FOS) is derived enzymatically from sucrose.

13. The composition as claimed in claim 12, wherein the fructooligosaccharides (FOS) is derived from enzymatic transfructosylation of sucrose.

14. The composition as claimed in any one of claims 10 to 13, wherein the fructooligosaccharides (FOS) has a total number of fructose or glucose units in the range of about 2 and about 8.

15. The composition as claimed in claim 14, wherein the fructooligosaccharides (FOS) has a total number of fructose or glucose units in the range of about 3 and about 5.

16. The composition as claimed in any one of claims 10 to 15, wherein the fructooligosaccharides (FOS) has a purity of about 95% or more.

17. The composition as claimed in any preceding claim, wherein the soluble fibre comprises glucomannan, beta glucan, pectin, gums, psyllium, or algin

18. The composition as claimed in any preceding claim, wherein the soluble fibre comprises glucomannan.

19. The composition as claimed in any preceding claim, wherein the chromium comprises trivalent chromium.

20. The composition as claimed in any preceding claim, wherein the chromium comprises one or more selected from: Chromium picolinate, Chromium chloride or Chromium nicotinate

21. The composition as claimed in either claim 18 or 20, wherein the composition comprises trivalent chromium in the range the range of about 10 μg to about 1000 μg.

22. The composition as claimed in any preceding claim, wherein the chromium has a particle size in the range of about 0.04 μm to about 2000 μm.

23. The composition as claimed in any preceding claim, wherein greater than about 90% of the soluble fibre has a particles size of about 125 μm or less.

24. The composition as claimed in any preceding claim, wherein the composition has a moisture content of about 7.5% or less.

25. The composition as claimed in claim 24, wherein the composition has a moisture content of about 5% or less.

26. The composition as claimed in any preceding claim, wherein the composition comprises a plurality of particles comprising a bulk density in the range of about 0.4 g/cc to about 0.7 g/cc.

27. The composition as claimed in claim 26, wherein the plurality of particles comprises a bulk density in the range of about 0.45 g/cc to about 0.65 g/cc.

28. The composition as claimed in any one of claims 26 to 27, wherein the particles have an average diameter of less than about 1.75 mm.

29. The composition as claimed in claim 28, wherein the particles have an average diameter of less than about 1.5 mm.

30. The composition as claimed in claim 29, wherein less than about 10% of the particles are about 1 mm or greater in diameter and/or less than about 10% of the particles are about 90 μm or less in diameter.

31. The composition as claimed in any preceding claim, for use in a food stuff, beverage, or as a food additive.

32. The composition as claimed in claim 31, for use as a dietary and/or weight loss supplement.

33. The composition as claimed in any one of claims 1 to 32, for use as a medicament.

34. The composition as claimed in any one of claims 1 to 30, for use in the weight management in an individual.

35. A method of producing a composition comprising prebiotic, chromium and soluble fibre components, wherein the composition is produced by mixing together prebiotic, chromium and soluble fibre components under conditions effective to form a homogeneous powder.

36. The method as claimed in claim 35, wherein the prebiotic, chromium and soluble fibre components are mixed under conditions so as to enable wet agglomeration of the components.

37. The method as claimed in either claim 35 or 36, wherein the prebiotic, chromium and soluble fibre components are mixed in a fluid bed agglomerator.

38. The method as claimed in either claim 36 or 37, wherein the prebiotic, chromium and soluble fibre components are wetted using water as the binding agent.

39. The method as claimed in claim 38, wherein the water is atomised.

40. The method as claimed in any one of claims 35 to 37, wherein the one or more particles comprise a homogenous mixture of the prebiotic, chromium and soluble fibre components.

41. The method as claimed in any one of claims 35 to 40, wherein the one or more particles are formed to have a diameter of about 2 mm or less.

42. The method as claimed in any one of claims 35 to 40, wherein the one or more particles are formed to have a diameter in the range of about 50 μm to about 2 mm.

43. The method as claimed in claim 42, wherein the one or more particles are formed to have a diameter in the range of about 90 μm to about 1 mm.

44. The method as claimed in any one of claims 35 to 43, wherein the one or more particles are dried so as to have a moisture content of about 7.5% or less.

45. The method as claimed in claim 44, wherein the one or more particles are dried so as to have a moisture content of about 5% or less.

46. The method as claimed in any one of claims 36 to 45, wherein the method produces a plurality of particles.

47. The method as claimed in claim 46, wherein the plurality of particles formed comprises a bulk density in the range of about 0.5 g/cc to about 0.7 g/cc.

48. The method as claimed in claim 47, wherein the plurality of particles comprises a bulk density in the range of about 0.55 g/cc to about 0.65 g/cc.48.

49. The method as claimed in any one of claims 35 to 48, wherein the prebiotics component comprises two or more selected from: inulin, fructooligosaccharides (FOS), galactooligosaccharides (GOS), α-gluco-oligosaccharides, and combinations thereof.

50. The method as claimed in claim 48, wherein the prebiotic component comprises fructooligosaccharides (FOS).

51. The method as claimed in claim 50, wherein the fructooligosaccharides (FOS) is derived from chicory or enzymatically from sucrose.

52. The composition as claimed in claim 51, wherein the fructooligosaccharides (FOS) is derived from enzymatic transfructosylation of sucrose

53. The method as claimed in any one of claims 50 to 52, wherein the fructooligosaccharides (FOS) has a total number of fructose or glucose units in the range of about 2 and about 8.

54. The method as claimed in claim 53, wherein fructooligosaccharides (FOS) has a total number of fructose or glucose units in the range of about 3 and about 5.

55. The method as claimed in any one of claims 50 to 54, wherein the fructooligosaccharides (FOS) has a purity of about 95% or more.

56. The method as claimed in any one of claims 35 to 55, wherein the chromium comprises trivalent chromium.

57. The method as claimed in claim 56, wherein the chromium comprises one or more selected from: Chromium picolinate, Chromium chloride or Chromium nicotinate

58. The method as claimed in any one of claims 35 to 57, for use in producing the composition as claimed in any one of claims 1 to 34.
